(19) United States
(12) Patent Application Publication
(10) Pub. No.: US 2009/0221592 A1
(43) Pub. Date: Sep. 3, 2009

(54) Dodecylsulfate Salt Of A Dipeptidyl Peptidase-Iv Inhibitor

(76) Inventors: Martha E. Ellison, Scotch Plains, NJ (US); Andrey V. Peresypkin, Cranford, NJ (US); Robert M. Wenslow, Cream Ridge, NJ (US)

Correspondence Address: MERCK AND CO., INC, P O BOX 2000, RAHWAY, NJ, 07065-0907, US

(21) Appl. No.: 11/988,197

(22) PCT Filed: Jul. 21, 2006
(86) PCT No.: PCT/US2006/028504

§ 371 Date: Jan. 2, 2008

Related U.S. Application Data
(60) Provisional application No. 60/702,232, filed on Jul. 25, 2005.

(51) Int. Cl. A61K 31/4985; C07D 487/04; A61P 3/10

(52) U.S. Cl. 514/249; 544/350

(57) ABSTRACT

The dodecylsulfate salt of (2R)-4-oxo-4-[3-(trifluoromethyl)-5,6-dihydro[1,2,4]triazolo[4,3-a]pyrazin-7(8H)-yl]-1-(2,4,5-trifluorophenyl)butan-2-amine is a potent inhibitor of dipeptidyl peptidase-IV and is useful for the treatment of Type 2 diabetes. The invention also relates to a crystalline anhydrate of the dodecylsulfate salt as well as a process for its preparation, pharmaceutical compositions containing this novel form and methods of use for the treatment of Type 2 diabetes, hyperglycemia, insulin resistance, and obesity.

### BACKGROUND OF THE INVENTION

Inhibition of dipeptidyl peptidase-IV (DPP-IV), an enzyme that inactivates both glucose-dependent insulinotropic peptide (GIP) and glucagon-like peptide 1 (GLP-1), represents a novel approach to the treatment and prevention of Type 2 diabetes, also known as non-insulin dependent diabetes mellitus (NIDDM). The therapeutic potential of DPP-IV inhibitors for the treatment of Type 2 diabetes has been reviewed: C. F. Deacon and J. J. Holst, “Dipeptidyl peptidase IV inhibition as an approach to the treatment and prevention of Type 2 diabetes: a historical perspective,” Biochem. Biophys. Res. Commun., 294: 1-4 (2000); K. Augustyns, et al., “Dipeptidyl peptidase IV inhibitors as new therapeutic-agents for the treatment of Type 2 diabetes,” Expert. Opin. Ther. Patents, 13: 499-510 (2003); D. J. Drucker, “Therapeutic potential of dipeptidyl peptidase IV inhibitors for the treatment of Type 2 diabetes,” Expert Opin. Investig. Drugs, 12: 87-100 (2003); M. A. Nauck et al., “Incretins and Their Analogues as New Antidiabetic Drugs,” Drug News Perspect., 16: 413-422 (2003); and O. A. Roges, et al., “The incretin effect and its potentiation by glucagon-like peptide 1-based therapies: a revolution in diabetes management,” Exp. Opin. Invest. Drugs, 14: 705-727 (2005).

WO 03/004498 (published 16 Jan. 2003) and U.S. Pat. No. 6,699,871 (issued 2 Mar. 2004), both assigned to Merck & Co., describe a class of beta-amino tetrahydrotriazolo[4,3-a]pyrazines, which are potent inhibitors of DPP-IV and therefore useful for the treatment of Type 2 diabetes. Specifically disclosed in WO 03/004498 and in the US patent is (2R)-4-oxo-4-[3-(trifluoromethyl)-5,6-dihydro[1,2,4]-triazolo[4,3-a]pyrazin-7(8H)-yl]-1-(2,4,5-trifluorophenyl)butan-2-amine (Compound I of structural formula I below). Pharmaceutically acceptable salts of this compound are generically encompassed within the scope of WO 03/004498.

However, there is no specific disclosure in WO 03/004498 and U.S. Pat. No. 6,699,871 of the newly discovered dodecylsulfate (laurylsulfate) salt of (2R)-4-oxo-4-[3-(trifluoromethyl)-5,6-dihydro[1,2,4]triazolo[4,3-a]pyrazin-7(8H)-yl]-1-(2,4,5-trifluorophenyl)butan-2-amine (Compound I).

### SUMMARY OF THE INVENTION

The present invention is concerned with a novel dodecylsulfate salt of the dipeptidyl peptidase-IV (DPP-IV) inhibitor (2R)-4-oxo-4-[3-(trifluoromethyl)-5,6-dihydro[1,2,4]triazolo[4,3-a]pyrazin-7(8H)-yl]-1-(2,4,5-trifluorophenyl)butan-2-amine and, in particular, a crystalline anhydrate form thereof. The dodecylsulfate salt of the present invention possesses properties that are advantageous in the preparation of pharmaceutical compositions of (2R)-4-oxo-4-[3-(trifluoromethyl)-5,6-dihydro[1,2,4]triazolo[4,3-a]pyrazin-7(8H)-yl]-1-(2,4,5-trifluorophenyl)butan-2-amine, such as chemical stability, ease of processing, handling, and dosing. In particular, it exhibits physicochemical properties, such as stability to stress, rendering it particularly suitable for the manufacture of various pharmaceutical dosage forms. The invention also concerns pharmaceutical compositions containing the novel dodecylsulfate salt of the present invention, in particular the crystalline anhydrate form, as well as methods for using them as DPP-IV inhibitors, in particular for the prevention or treatment of Type 2 diabetes, hyperglycemia, insulin resistance, and obesity.

### BRIEF DESCRIPTION OF THE DRAWINGS

FIG. 1 is a characteristic X-ray diffraction pattern of the crystalline dodecylsulfate salt anhydrate of Compound I of the present invention.

FIG. 2 is a carbon-13 cross-polarization magic-angle spinning (CPMAS) nuclear magnetic resonance (NMR) spectrum of the crystalline dodecylsulfate salt anhydrate of Compound I of the present invention.

FIG. 3 is a fluorine-19 magic-angle spinning (MAS) nuclear magnetic resonance (NMR) spectrum of the crystalline dodecylsulfate salt anhydrate of Compound I of the present invention.

FIG. 4 is a typical thermogravimetric analysis (TGA) curve of the crystalline dodecylsulfate salt anhydrate of Compound I of the present invention.

FIG. 5 is a typical differential scanning calorimetry (DSC) curve of the crystalline dodecylsulfate salt anhydrate of Compound I of the present invention.

### DETAILED DESCRIPTION OF THE INVENTION

This invention provides a novel dodecylsulfate salt of (2R)-4-oxo-4-[3-(trifluoromethyl)-5,6-dihydro[1,2,4]triazolo[4,3-a]pyrazin-7(8H)-yl]-1-(2,4,5-trifluorophenyl)butan-2-amine of structural formula I (Compound I):

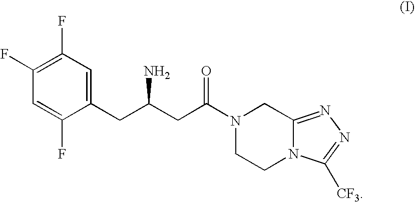

The dodecylsulfate salt of the present invention is represented by structural formula II:

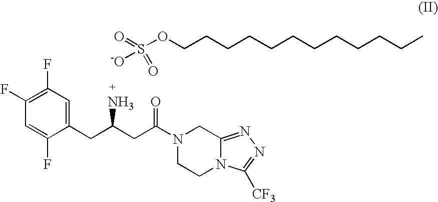

More specifically, the dodecylsulfate salt of the present invention is comprised of one molar equivalent of mono-protonated (2R)-4-oxo-4-[3-(trifluoromethyl)-5,6-dihydro[1,2,4]triazolo[4,3-a]pyrazin-7(8H)-yl]-1-(2,4,5-trifluorophenyl)butan-2-amine cation and one molar equivalent of dodecylsulfate (laurylsulfate) anion.

In one embodiment of the present invention, the dodecylsulfate salt of Compound I is in the form of a crystalline anhydrate.

Another embodiment of the present invention provides a particular salt drug substance that comprises the crystalline dodecylsulfate salt of the present invention present in a detectable amount. By “drug substance” is meant the active pharmaceutical ingredient. The amount of crystalline dodecylsulfate salt in the drug substance can be quantified by the use of physical methods such as X-ray powder diffraction, solid-state fluorine-19 magic-angle spinning (MAS) nuclear magnetic resonance spectroscopy, solid-state carbon-13 cross-polarization magic-angle spinning (CPMAS) nuclear magnetic resonance spectroscopy, solid state Fourier-transform infrared spectroscopy, and Raman spectroscopy. In a class of this embodiment, about 5% to about 100% by weight of the crystalline dodecylsulfate salt of the present invention is present in the drug substance. In a second class of this embodiment, about 10% to about 100% by weight of the crystalline dodecylsulfate salt is present in the drug substance. In a third class of this embodiment, about 25% to about 100% by weight of the crystalline dodecylsulfate salt is present in the drug substance. In a fourth class of this embodiment, about 50% to about 100% by weight of the crystalline dodecylsulfate salt is present in the drug substance. In a fifth class of this embodiment, about 75% to about 100% by weight of the crystalline dodecylsulfate salt is present in the drug substance. In a sixth class of this embodiment, substantially all of the salt drug substance is the crystalline dodecylsulfate salt of the present invention, i.e., the salt drug substance is substantially phase pure crystalline dodecylsulfate salt.

The crystalline dodecylsulfate salt of the present invention exhibits pharmaceutic advantages over the free base and the previously disclosed amorphous hydrochloric acid salt (WO 03/004498) in the preparation of a pharmaceutical drug product containing the pharmacologically active ingredient. In particular, the chemical and physical stability of the crystalline dodecylsulfate salt constitute advantageous properties in the preparation of solid pharmaceutical dosage forms containing the pharmacologically active ingredient.

The dodecylsulfate salt of the present invention, which exhibits potent DPP-IV inhibitory properties, is particularly useful for the prevention or treatment of Type 2 diabetes, hyperglycemia, insulin resistance, and obesity.

Another aspect of the present invention provides a method for the prevention or treatment of clinical conditions for which an inhibitor of DPP-IV is indicated, which method comprises administering to a patient in need of such prevention or treatment a prophylactically or therapeutically effective amount of the dodecylsulfate salt of the present invention, in particular the crystalline anhydrate form thereof. Such clinical conditions include diabetes, in particular Type 2 diabetes, hyperglycemia, insulin resistance, and obesity.

The present invention also provides for the use of the dodecylsulfate salt of Compound I of the present invention, in particular the crystalline anhydrate form, for the prevention or treatment in a mammal of clinical conditions for which an inhibitor of DPP-IV is indicated, in particular Type 2 diabetes, hyperglycemia, insulin resistance, and obesity.

The present invention also provides for the use of the dodecylsulfate salt of Compound I of the present invention, in particular the crystalline anhydrate form, for the manufacture of a medicament for the prevention or treatment in a mammal of clinical conditions for which an inhibitor of DPP-IV is indicated, in particular Type 2 diabetes, hyperglycemia, insulin resistance, and obesity.

The present invention also provides pharmaceutical compositions comprising the dodecylsulfate salt of the present invention, in particular the crystalline anhydrate form, in association with one or more pharmaceutically acceptable carriers or excipients. In one embodiment the pharmaceutical composition comprises a therapeutically effective amount of the active pharmaceutical ingredient in admixture with pharmaceutically acceptable excipients wherein the active pharmaceutical ingredient comprises a detectable amount of the crystalline dodecylsulfate salt of the present invention. In a second embodiment the pharmaceutical composition comprises a therapeutically effective amount of the active pharmaceutical ingredient in admixture with pharmaceutically acceptable excipients wherein the active pharmaceutical ingredient comprises about 5% to about 100% by weight of the crystalline dodecylsulfate salt of the present invention. In a class of this second embodiment, the active pharmaceutical ingredient in such compositions comprises about 10% to about 100% by weight of the crystalline dodecylsulfate salt. In a second class of this embodiment, the active pharmaceutical ingredient in such compositions comprises about 25% to about 100% by weight of the crystalline dodecylsulfate salt. In a third class of this embodiment, the active pharmaceutical ingredient in such compositions comprises about 50% to about 100% by weight of the crystalline dodecylsulfate salt. In a fourth class of this embodiment, the active pharmaceutical ingredient in such compositions comprises about 75% to about 100% by weight of the crystalline dodecylsulfate salt. In a fifth class of this embodiment, substantially all of the active pharmaceutical ingredient is the crystalline dodecylsulfate salt of the present invention, i.e., the active pharmaceutical ingredient is substantially phase pure crystalline dodecylsulfate salt.

The compositions in accordance with the invention are suitably in unit dosage forms such as tablets, pills, capsules, powders, granules, sterile solutions or suspensions, metered aerosol or liquid sprays, drops, ampoules, auto-injector devices or suppositories. The compositions are intended for oral, parenteral, intranasal, sublingual, or rectal administration, or for administration by inhalation or insufflation. Formulation of the compositions according to the invention can conveniently be effected by methods known from the art, for example, as described in Remington's Pharmaceutical Sciences, 17^{th} ed., 1995.

The dosage regimen is selected in accordance with a variety of factors including type, species, age, weight, sex and medical condition of the patient; the severity of the condition to be treated; the route of administration; and the renal and hepatic function of the patient. An ordinarily skilled physician, veterinarian, or clinician can readily determine and prescribe the effective amount of the drug required to prevent, counter or arrest the progress of the condition.

Oral dosages of the present invention, when used for the indicated effects, will range between about 0.01 mg per kg of body weight per day (mg/kg/day) to about 100 mg/kg/day, preferably 0.01 to 10 mg/kg/day, and most preferably 0.1 to 5.0 mg/kg/day. For oral administration, the compositions are preferably provided in the form of tablets containing 0.01, 0.05, 0.1, 0.5, 1.0, 2.5, 5.0, 10.0, 15.0, 25.0, 50.0, 100 and 500 milligrams of the active ingredient for the symptomatic adjustment of the dosage to the patient to be treated. A medicament typically contains from about 0.01 mg to about 500 mg of the active ingredient, preferably, from about 1 mg to about 200 mg of active ingredient. Intravenously, the most preferred doses will range from about 0.1 to about 10 mg/kg/minute during a constant rate infusion. Advantageously, the dodecylsulfate salt of the present invention may be administered in a single daily dose, or the total daily dosage may be administered in divided doses of two, three or four times daily. Furthermore, the dodecylsulfate salt of the present invention can be administered in intranasal form via topical use of suitable intranasal vehicles, or via transdermal routes, using those forms of transdermal skin patches well known to those of ordinary skill in the art. To be administered in the form of a transdermal delivery system, the dosage administration will, of course, be continuous rather than intermittent throughout the dosage regimen.

In the methods of the present invention, the dodecylsulfate salt described herein in detail can form the active pharmaceutical ingredient, and is typically administered in admixture with suitable pharmaceutical diluents, excipients or carriers (collectively referred to herein as ‘carrier’ materials) suitably selected with respect to the intended form of administration, that is, oral tablets, capsules, elixirs, syrups and the like, and consistent with conventional pharmaceutical practices.

For instance, for oral administration in the form of a tablet or capsule, the active drug component can be combined with an oral, non-toxic, pharmaceutically acceptable, inert carrier such as lactose, starch, sucrose, glucose, methyl cellulose, magnesium stearate, dicalcium phosphate, calcium sulfate, mannitol, sorbitol and the like; for oral administration in liquid form, the oral drug component can be combined with any oral, non-toxic, pharmaceutically acceptable inert carrier such as ethanol, glycerol, water and the like. Moreover, when desired or necessary, suitable binders, lubricants, disintegrating agents and coloring agents can also be incorporated into the mixture. Suitable binders include starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms include sodium oleate, sodium stearate, magnesium stearate, sodium benzoate, sodium acetate, sodium chloride and the like. Disintegrators include, without limitation, starch, methyl cellulose, agar, bentonite, xanthan gum and the like.

In a still further aspect, the present invention provides a method for the treatment and/or prevention of clinical conditions for which a DPP-IV inhibitor is indicated, which method comprises administering to a patient in need of such prevention or treatment a prophylactically or therapeutically effective amount of the dodecylsulfate salt of Compound I as defined above or, in particular, the crystalline anhydrate form thereof, in combination with another agent useful for the treatment of Type 2 diabetes, hyperglycemia, insulin resistance, and obesity. Such agents include:

(a) insulin sensitizers including (i) PPARγ agonists, such as the glitazones (e.g. troglitazone, pioglitazone, englitazone, MCC-555, rosiglitazone, balaglitazone, and the like) and other PPAR ligands, including PPARα/γ dual agonists, such as KRP-297, muraglitazar, naveglitazar, tesaglitazar, TAK-559, PPARα agonists, such as fenofibric acid derivatives (gemfibrozil, clofibrate, fenofibrate and bezafibrate), and selective PPARγ modulators (SPPARγM's), such as disclosed in WO 02/060388, WO 02/08188, WO 2004/019869, WO 2004/020409, WO 2004/020408, and WO 2004/066963; (ii) biguanides such as metformin and phenformin, and (iii) protein tyrosine phosphatase-1B (PTP-1B) inhibitors;

(b) insulin or insulin mimetics;

(c) sulfonylureas and other insulin secretagogues, such as tolbutamide, glyburide, glipizide, glimepiride, and meglitinides, such as nateglinide and repaglinide;

(d) α-glucosidase inhibitors (such as acarbose and miglitol);

(e) glucagon receptor antagonists, such as those disclosed in WO 97/16442; WO 98/04528, WO 98/21957; WO 98/22108; WO 98/22109; WO 99/01423, WO 00/39088, and WO 00/69810; WO 2004/050039; and WO 2004/069158;

(f) GLP-1, GLP-1 analogues or mimetics, and GLP-1 receptor agonists, such as exendin-4 (exenatide), liraglutide (N,N-2211), CJC-1131, LY-307161, and those disclosed in WO 00/42026 and WO 00/59887;

(g) GIP and GIP mimetics, such as those disclosed in WO 00/58360, and GIP receptor agonists;

(h) PACAP, PACAP mimetics, and PACAP receptor agonists such as those disclosed in WO 01/23420;

(i) cholesterol lowering agents such as (i) HMG-CoA reductase inhibitors (lovastatin, simvastatin, pravastatin, cerivastatin, fluvastatin, atorvastatin, itavastatin, and rosuvastatin, and other statins), (ii) sequestrants (cholestyramine, colestipol, and dialkylaminoalkyl derivatives of a cross-linked dextran), (iii) nicotinyl alcohol, nicotinic acid or a salt thereof, (iv) PPARα agonists such as fenofibric acid derivatives (gemfibrozil, clofibrate, fenofibrate and bezafibrate), (v) PPARα/γ dual agonists, such as naveglitazar and muraglitazar, (vi) inhibitors of cholesterol absorption, such as beta-sitosterol and ezetimibe, (vii) acyl CoA:cholesterol acyltransferase inhibitors, such as avasimibe, and (viii) antioxidants, such as probucol;

(j) PPARδ agonists, such as those disclosed in WO 97/28149;

(k) antiobesity compounds, such as fenfluramine, dexfenfluramine, phentermine, sibutramine, orlistat, neuropeptide Y₁ or Y₅ antagonists, CB1 receptor inverse agonists and antagonists, β₃ adrenergic receptor agonists, melanocortin-receptor agonists, in particular melanocortin-4 receptor agonists, ghrelin antagonists, bombesin receptor agonists (such as bombesin receptor subtype-3 agonists), cholecystokinin 1 (CCK-1) receptor agonists, and melanin-concentrating hormone (MCH) receptor antagonists;

(l) ileal bile acid transporter inhibitors;

(m) agents intended for use in inflammatory conditions such as aspirin, non-steroidal anti-inflammatory drugs (NSAIDs), glucocorticoids, azulfidine, and selective cyclooxygenase-2 (COX-2) inhibitors;

(n) antihypertensive agents, such as ACE inhibitors (enalapril, lisinopril, captopril, quinapril, tandolapril), A-II receptor blockers (losartan, candesartan, irbesartan, valsartan, telmisartan, and eprosartan), beta blockers and calcium channel blockers;

(o) glucokinase activators (GKAs), such as those disclosed in WO 03/015774; WO 04/076420; and WO 04/081001;

(p) inhibitors of 11β-hydroxysteroid dehydrogenase type 1, such as those disclosed in U.S. Pat. No. 6,730,690; WO 03/104207; and WO 04/058741;

(q) inhibitors of cholesteryl ester transfer protein (CETP), such as torcetrapib; and

(r) inhibitors of fructose 1,6-bisphosphatase, such as those disclosed in U.S. Pat. Nos. 6,054,587; 6,110,903; 6,284,748; 6,399,782; and 6,489,476.

Compounds described herein may exist as tautomers such as keto-enol tautomers. The individual tautomers as well as mixtures thereof are encompassed with compounds of structural formula I.

The term “% enantiomeric excess” (abbreviated “ee”) shall mean the % major enantiomer less the % minor enantiomer. Thus, a 70% enantiomeric excess corresponds to formation of 85% of one enantiomer and 15% of the other. The term “enantiomeric excess” is synonymous with the term “optical purity.”

According to a further aspect, the present invention provides a process for the preparation of the dodecylsulfate salt of Compound I of the present invention, which process comprises treating a solution of the phosphate salt of (2R)-4-oxo-4-[3-(trifluoromethyl)-5,6-dihydro[1,2,4]-triazolo[4,3-a]pyrazin-7(8H)-yl]-1-(2,4,5-trifluorophenyl)butan-2-amine (Compound I):

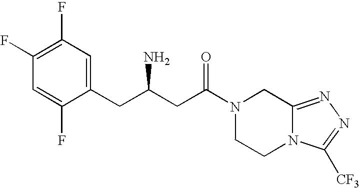

in a suitable solvent with sodium dodecylsulfate (sodium laurylsulfate). The process is carried out generally at about 0° C. to about 100° C., and preferably at about 20° C. to about 60° C. Generally, the solvent is water or an organic solvent, such as a linear or branched C₁-C₄ alkanol, such as methanol, ethanol, or isopropanol, a linear or branched C₁₋₄ alkyl acetate, such as ethyl acetate or isopropyl acetate, diethyl ether, tetrahydrofuran, toluene, acetone, and acetonitrile. A mixture of water and an organic solvent may also be employed. Crystallization is then effected by cooling the mixture and optional seeding with crystals of the authentic dodecylsulfate salt, but the latter is not essential. The dodecylsulfate salt is then isolated by filtration and drying.

The phosphate salt of Compound I can be prepared by the procedures detailed in Schemes 1 and 2 below.

### Preparation of 3-(trifluoromethyl)-5,6,7,8-tetrahydro[1,2,4]triazolo[4,3-a]pyrazine hydrochloric acid (1-4)

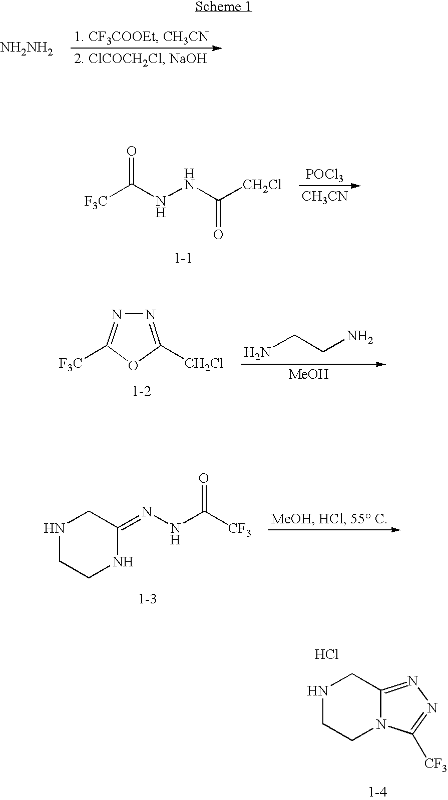

### Step A: Preparation of bishydrazide (1-1)

Hydrazine (20.1 g, 35 wt % in water, 0.22 mol) was mixed with 310 mL of acetonitrile. 31.5 g of ethyl trifluoroacetate (0.22 mol) was added over 60 min. The internal temperature was increased to 25° C. from 14° C. The resulting solution was aged at 22-25° C. for 60 min. The solution was cooled to 7° C. 17.9 g of 50 wt % aqueous NaOH (0.22 mol) and 25.3 g of chloroacetyl chloride (0.22 mol) were added simultaneously over 130 min at a temperature below 16° C. When the reaction was complete, the mixture was vacuum distilled to remove water and ethanol at 27˜30° C. and under 26˜27 in Hg vacuum. During the distillation, 720 mL of acetonitrile was added slowly to maintain constant volume (approximately 500 mL). The slurry was filtered to remove sodium chloride. The cake was rinsed with about 100 mL of acetonitrile. Removal of the solvent afforded bis-hydrazide 1-1 (94.4 area % pure by HPLC assay).

¹H-NMR (400 MHz, DMSO-d₆): δ 4.2 (s, 2H), 10.7 (s, 1H), and 11.6 (s, 1H) ppm.

¹³C-NMR (100 MHz, DMSO-d₆): δ 41.0, 116.1 (q, J=362 Hz), 155.8 (q, J=50 Hz), and 165.4 ppm.

### Step B: Preparation of 5-(trifluoromethyl)-2-(chloromethyl)-1,3,4-oxadiazole (1-2)

Bishydrazide 1-1 from Step A (43.2 g, 0.21 mol) in ACN (82 mL) was cooled to 5° C. Phosphorus oxychloride (32.2 g, 0.21 mol) was added, maintaining the temperature below 10° C. The mixture was heated to 80° C. and aged at this temperature for 24 h until HPLC showed less than 2 area % of 1-1. In a separate vessel, 260 mL of IPAc and 250 mL of water were mixed and cooled to 0° C. The reaction slurry was charged to the quench keeping the internal temperature below 10° C. After the addition, the mixture was agitated vigorously for 30 min, the temperature was increased to room temperature and the aqueous layer was cut. The organic layer was then washed with 215 mL of water, 215 mL of 5 wt % aqueous sodium bicarbonate and finally 215 mL of 20 wt % aqueous brine solution. HPLC assay yield after work up was 86-92%. Volatiles were removed by distillation at 75-80 mm Hg, 55° C. to afford an oil which could be used directly in Step C without further purification. Otherwise the product can be purified by distillation to afford 1-2.

¹H-NMR (400 MHz, CDCl₃): δ 4.8 (s, 2H) ppm.

¹³C-NMR (100 MHz, CDCl₃): δ 32.1, 115.8 (q, J=337 Hz), 156.2 (q, J=50 Hz), and 164.4 ppm.

### Step C: Preparation of N-[(2Z)-piperazin-2-ylidene]trifluoroacetohvdrazide (1-3)

To a solution of ethylenediamine (33.1 g, 0.55 mol) in methanol (150 mL) cooled at −20° C. was added distilled oxadiazole 1-2 from Step B (29.8 g, 0.16 mol) while keeping the internal temperature at −20° C. After the addition was complete, the resulting slurry was aged at −20° C. for 1 h. Ethanol (225 mL) was then charged and the slurry slowly warmed to −5° C. After 60 min at −5° C., the slurry was filtered and washed with ethanol (60 mL) at −5° C. Amidine 1-3 was obtained as a white solid (99.5 area wt % pure by HPLC).

¹H-NMR (400 MHz, DMSO-d₆): δ 2.9 (t, 2H), 3.2 (t, 2H), 3.6 (s, 2H), and 8.3 (b, 1H) ppm. ¹³C-NMR (100 MHz, DMSO-d₆): δ 40.8, 42.0, 43.3, 119.3 (q, J=350 Hz), 154.2, and 156.2 (q, J=38 Hz) ppm.

### Step D: Preparation of 3-(trifluoromethyl)-5,6,7,8-tetrahydro[1,2,4]triazolo[4,3-a]pyrazine hydrochloric acid (1-4)

A suspension of amidine 1-3 (27.3 g, 0.13 mol) in 110 mL of methanol was warmed to 55° C. 37% Hydrochloric acid (11.2 mL, 0.14 mol) was added over 15 min at this temperature. During the addition, all solids dissolved resulting in a clear solution. The reaction was aged for 30 min. The solution was cooled down to 20° C. and aged at this temperature until a seed bed formed (10 min to 1 h). 300 mL of MTBE was charged at 20° C. over 1 h. The resulting slurry was cooled to 2° C., aged for 30 min and filtered. Solids were washed with 50 mL of ethanol:MTBE (1:3) and dried under vacuum at 45° C. (99.5 area wt % pure by HPLC).

¹H-NMR (400 MHz, DMSO-d₆): δ 3.6 (t, 2H), 4.4 (t, 2H), 4.6 (s, 2H), and 10.6 (b, 2H) ppm; ¹³C-NMR (100 MHz, DMSO-d₆): δ: 39.4, 39.6, 41.0, 118.6 (q, J=325 Hz), 142.9 (q, J=50 Hz), and 148.8 ppm.

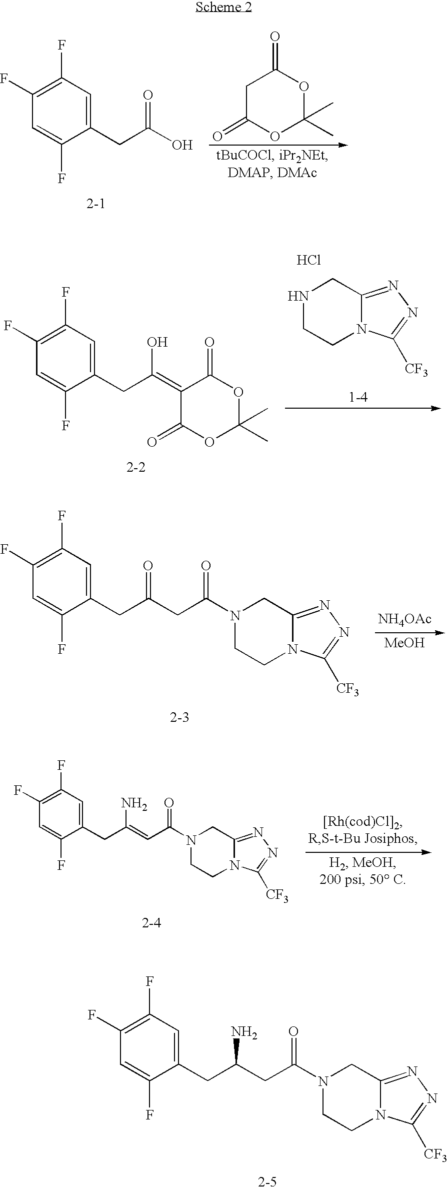

### Step A: Preparation of 4-oxo-4-[3-(trifluoromethyl)-5,6-dihydro[1,2,4]triazolo[4,3-a]pyrazin-7(8H)-yl]-1-(2,4,5-trifluorophenyl)butan-2-one (2-3)

2,4,5-Trifluorophenylacetic acid (2-1) (150 g, 0.789 mol), Meldrum's acid (125 g, 0.868 mol), and 4-(dimethylamino)pyridine (DMAP) (7.7 g, 0063 mol) were charged into a 5 L three-neck flask. N,N-Dimethylacetamide (DMAc) (525 mL) was added in one portion at room temperature to dissolve the solids. N,N-diisopropylethylamine (282 mL, 1.62 mol) was added in one portion at room temperature while maintaining the temperature below 40° C. Pivaloyl chloride (107 mL, 0.868 mol) was added dropwise over 1 to 2 h while maintaining the temperature between 0 and 5° C. The reaction mixture was aged at 5° C. for 1 h. Triazole hydrochloric acid 1-4 (180 g, 0.789 mol) was added in one portion at 40-50° C. The reaction solution was aged at 70° C. for several h. 5% Aqueous sodium hydrogencarbonate solution (625 mL) was then added dropwise at 20-45° C. The batch was seeded and aged at 20-30° C. for 1-2 h. Then an additional 525 mL of 5% aqueous sodium hydrogencarbonate solution was added dropwise over 2-3 h. After aging several h at room temperature, the slurry was cooled to 0-5° C. and aged 1 h before filtering the solid. The wet cake was displacement-washed with 20% aqueous DMAc (300 mL), followed by an additional two batches of 20% aqueous DMAc (400 mL), and finally water (400 mL). The cake was suction-dried at room temperature.

### Step B: Preparation of (2Z)-4-oxo-4-[3-(trifluoromethyl)-5,6-dihydro[1,2,4]triazolo[4,3-a]pyrazin-7(8H)-1-(2,4,5-trifluorophenyl)but-2-en-2-amine (2-4)

A 5 L round-bottom flask was charged with methanol (100 mL), the ketoamide 2-3 (200 g), and ammonium acetate (110.4 g). Methanol (180 mL) and 28% aqueous ammonium hydroxide (58.6 mL) were then added keeping the temperature below 30° C. during the addition. Additional methanol (100 mL) was added to the reaction mixture. The mixture was heated at reflux temperature and aged for 2 h. The reaction was cooled to room temperature and then to about 5° C. in an ice-bath. After 30 min, the solid was filtered and dried to afford 2-4 as a solid; m.p. 271.2° C.

### Step C: Preparation of (2R)-4-oxo-4-[3-(trifluoromethyl)-5,6-dihydro[1,2,4]triazolo[4,3-a]pyrazin-7(8H)-yl]-1-(2,4,5-trifluorophenyl)butan-2-amine (2-5)

Into a 500 ml flask were charged chloro(1,5-cyclooctadiene)rhodium(I) dimer {[Rh(cod)Cl]₂} (292 mg, 1.18 mmol) and (R,S) t-butyl Josiphos (708 mg, 1.3 mmol) under a nitrogen atmosphere. Degassed MeOH was then added (200 mL) and the mixture was stirred at room temperature for 1 h. Into a 4 L hydrogenator was charged the enamine amide 2-4 (118 g, 0.29 mol) along with MeOH (1 L). The slurry was degassed. The catalyst solution was then transferred to the hydrogenator under nitrogen. After degassing three times, the enamine amide was hydrogenated under 200 psi hydrogen gas at 50° C. for 13 h. Assay yield was determined by HPLC to be 93% and optical purity to be 94% ee.

The optical purity was further enhanced in the following manner. The methanol solution from the hydrogenation reaction (18 g in 180 mL MeOH) was concentrated and switched to methyl t-butyl ether (MTBE) (45 mL). Into this solution was added aqueous H₃PO₄ solution (0.5 M, 95 mL). After separation of the layers, 3N NaOH (35 mL) was added to the water layer, which was then extracted with MTBE (180 mL+100 mL). The MTBE solution was concentrated and solvent switched to hot toluene (180 mL, about 75° C.). The hot toluene solution was then allowed to cool to 0° C. slowly (5-10 h). The crystals were isolated by filtration (98-99% ee); m.p. 114.1-115.7° C.

¹H NMR (300 MHz, CD₃CN): δ 7.26 (m), 7.08 (m), 4.90 (s), 4.89 (s), 4.14 (m), 3.95 (m), 3.40 (m), 2.68 (m), 2.49 (m), 1.40 (bs).

Compound 2-5 exists as amide bond rotamers. Unless indicated, the major and minor rotamers are grouped together since the carbon-13 signals are not well resolved:

¹³C NMR (CD₃CN): δ 171.8, 157.4 (ddd, J_{CF}=242.4, 9.2, 2.5 Hz), 152.2 (major), 151.8 (minor), 149.3 (ddd; J_{CF}=246.7, 14.2, 12.9 Hz), 147.4 (ddd, J_{CF}=241.2, 12.3, 3.7 Hz), 144.2 (q, J_{CF}=38.8 Hz), 124.6 (ddd, J_{CF}=18.5, 5.9, 4.0 Hz), 120.4 (dd, J_{CF}=19.1, 6.2 Hz), 119.8 (q, J_{CF}=268.9 Hz), 106.2 (dd, J_{CF}=29.5, 20.9 Hz), 50.1, 44.8, 44.3 (minor), 43.2 (minor), 42.4, 41.6 (minor), 41.4, 39.6, 38.5 (minor), 36.9.

The following high-performance liquid chromatographic (HPLC) conditions were used to determine percent conversion to product:

| | |
|---|---|
| Column: | Waters Symmetry C18, 250 mm × 4.6 mm |
| Eluent: | Solvent A: 0.1 vol % HClO₄/H₂O |
| | Solvent B: acetonitrile |
| Gradient: | 0 min 75% A:25% B |
| | 10 min 25% A:75% B |
| | 12.5 min 25% A:75% B |
| | 15 min 75% A:25% B |
| Flow rate: | 1 mL/min |
| Injection Vol.: | 10 μL |
| UV detection: | 210 nm |
| Column temp.: | 40° C. |
| Retention times: | compound 2-4: 9.1 min |
| | compound 2-5: 5.4 min |
| | tBu Josiphos: 8.7 min |

The following high-performance liquid chromatographic (HPLC) conditions were used to determine optical purity:

| | |
|---|---|
| Column: | Chirapak, AD-H, 250 mm × 4.6 mm |
| Eluent: | Solvent A: 0.2 vol. % diethylamine in heptane |
| | Solvent B: 0.1 vol % diethylamine in ethanol |
| Isochratic Run Time: | 18 min |
| Flow rate: | 0.7 mL/min |
| Injection Vol.: | 7 μL |
| UV detection: | 268 nm |
| Column temp.: | 35° C. |
| Retention times: | (R)-amine 2-5: 13.8 min |
| | (S)-amine 2-5: 11.2 min |

### (2R)-4-oxo-4-[3-(trifluoromethyl)-5,6-dihydro[1,2,4]triazolo[4,3-a]pyrazin-7(8H)-yl]-1-(2,4,5-trifluorophenyl)butan-2-amine phosphate salt

A 250 mL round bottom flask equipped with an overhead stirrer, heating mantle and thermocouple, was charged with 31.5 mL of isopropanol (EPA), 13.5 mL water, 15.0 g (36.9 mmol) of (2R)-4-oxo-4-[3-(trifluoromethyl)-5,6-dihydro[1,2,4]triazolo[4,3-a]pyrazin-7(8H)-yl]-1-(2,4,5-trifluorophenyl)butan-2-amine freebase and 4.25 g (36.9 mmol) of 85% aqueous phosphoric acid. The mixture was heated to 75° C. A thick white precipitate formed at lower temperatures but dissolved upon reaching 75° C. The solution was cooled to 68° C. and then held at that temperature for 2 h. A slurry bed of solids formed during this age time [the solution can be seeded with 0.5 to 5 wt % of small particle size (alpine milled) monohydrate]. The slurry was then cooled at a rate of 4° C./h to 21° C. and then held overnight. 105 mL of IPA was then added to the slurry. After 1 h the slurry was filtered and washed with 45 mL IPA (solids can also be washed with a water/IPA solution to avoid turnover to other crystal forms). The solids were dried on the frit with open to air.

### (2R)-4-oxo-4-[3-(trifluoromethyl)-5,6-dihydro[1,2,4]triazolo[4,3-a]pyrazin-7(8H)-yl]-1-(2,4,5-trifluorophenyl)butan-2-amine dodecylsulfate salt anhydrate

Compound I phosphate salt was dissolved in water at a concentration of approximately 10 mg/mL. The mixture was agitated until no solid material was apparent. Two molar equivalents of sodium dodecylsulfate were added to the solution. The solution was then stirred vigorously with a magnetic stir bar on a stir plate. The solution was initially clear, but after approximately 5 minutes, solid material was present. The solid material was vacuum filtered, washed with water, and pulled under vacuum for 24 hours at 40° C. to remove the water and leave a white crystalline solid.

X-ray powder diffraction studies are widely used to characterize molecular structures, crystallinity, and polymorphism. The X-ray powder diffraction pattern of the crystalline dodecylsulfate salt of the present invention was generated on a Philips Analytical X'Pert PRO X-ray Diffraction System with PW3040/60 console. A PW3373/00 ceramic Cu LEF X-ray tube K-Alpha radiation was used as the source.

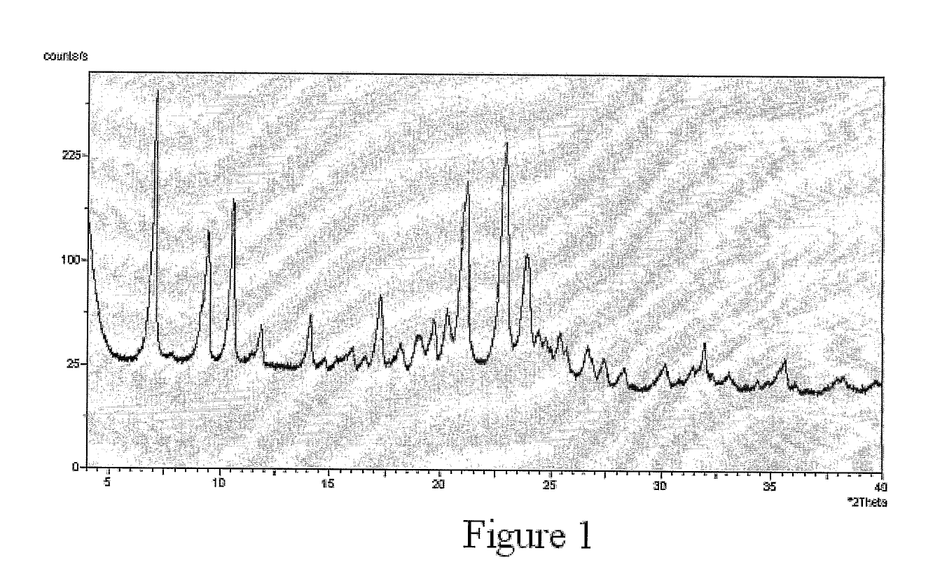

FIG. 1 shows the X-ray diffraction pattern for the crystalline dodecylsulfate salt anhydrate of Compound I of the present invention. The salt exhibited characteristic diffraction peaks corresponding to 2-theta values of 7.09, 9.46, 10.6, 11.90, 14.11, 17.28, 21.17, 22.92, and 23.87 degrees.

In addition to the X-ray powder diffraction patterns described above, the dodecylsulfate salt of the present invention was further characterized by solid-state carbon-13 and fluorine-19 nuclear magnetic resonance (NMR) spectroscopy. The solid-state carbon-13 NMR spectrum was obtained on a Bruker DSX 400WB NMR system using a Bruker 4 mm double resonance CPMAS probe. The carbon-13 NMR spectrum utilized proton/carbon-13 cross-polarization magic-angle spinning with variable-amplitude cross polarization. The sample was spun at 12.0 kHz, and a total of 1024 scans were collected with a recycle delay of 10 seconds. A line broadening of 20 Hz was applied to the spectrum before FT was performed. Chemical shifts are reported on the TMS scale using the carbonyl carbon of glycine (176.03 p.p.m.) as a secondary reference.

The solid-state fluorine-19 NMR spectrum was obtained on a Bruker DSX 400WB NMR system using a Bruker 4 mm CRAMPS probe. The NMR spectrum utilized a simple pulse-acquire pulse program. The samples were spun at 15.0 kHz, and a total of 128 scans were collected with a recycle delay of 5 seconds. A vespel endcap was utilized to minimize fluorine background. A line broadening of 100 Hz was applied to the spectrum before FT was performed. Chemical shifts are reported using poly(tetrafluoroethylene) (teflon) as an external secondary reference which was assigned a chemical shift of −122 ppm.

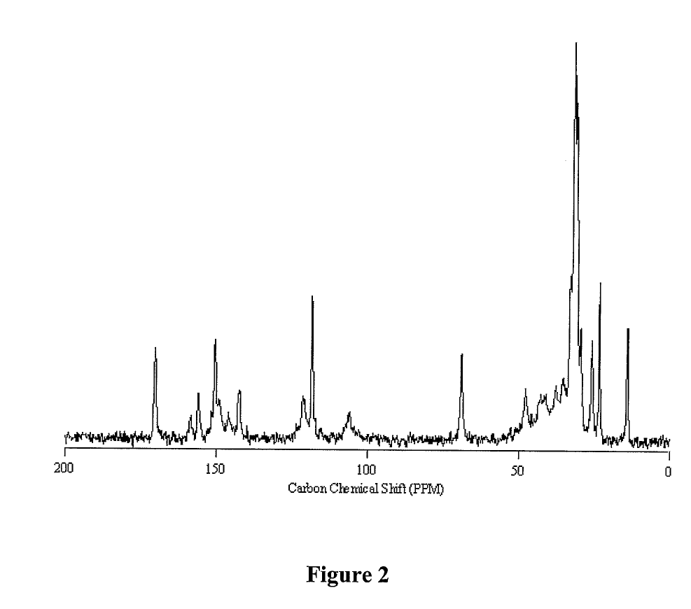

FIG. 2 shows the solid-state carbon-13 CPMAS NMR spectrum for the crystalline dodecylsulfate salt anhydrate of Compound I of the present invention. The salt exhibited characteristic signals with chemical shift values of 14.0, 31.8, and 69.0 p.p.m. Further characteristic of the anhydrate form are the signals with chemical shift values of 118.3, 150.5, and 170.2 p.p.m.

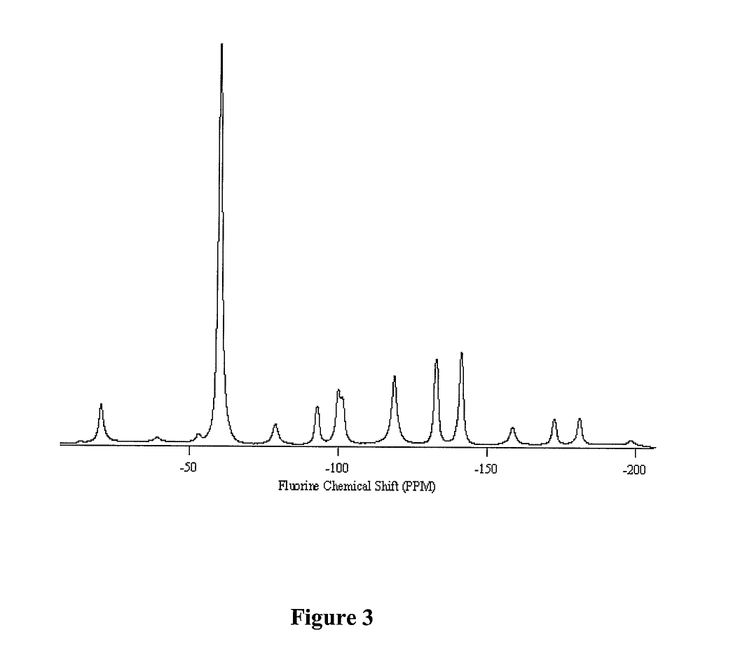

FIG. 3 shows the solid-state fluorine-19 MAS NMR spectrum for the crystalline dodecylsulfate salt anhydrate of Compound I of the present invention. The salt exhibited characteristic signals with chemical shift values of −60.1, −118.7, and −141.3 p.p.m. Further characteristic of the anhydrate form are the signals with chemical shift values of −132.9, −93.0, and −20.3 p.p.m.

The crystalline dodecylsulfate salt of Compound I of the present invention was further characterized by means of its differential scanning calorimetry (DSC) curve and its thermogravimetric analysis (TGA) curve.

A TA Instruments DSC 2910 or equivalent instrumentation was used to obtain the DSC curves. Between 2 and 6 mg sample was weighed into an open pan. This pan was then crimped and placed at the sample position in the calorimeter cell. An empty pan was placed at the reference position. The calorimeter cell was closed and a flow of nitrogen was passed through the cell. The heating program was set to heat the sample at a heating rate of 10° C./min to a temperature of approximately 250° C. The heating program was started. When the run was completed, the data were analyzed using the DSC analysis program contained in the system software. The melting endotherm was integrated between baseline temperature points that are above and below the temperature range over which the endotherm was observed. The data reported are the onset temperature, peak temperature, and enthalpy.

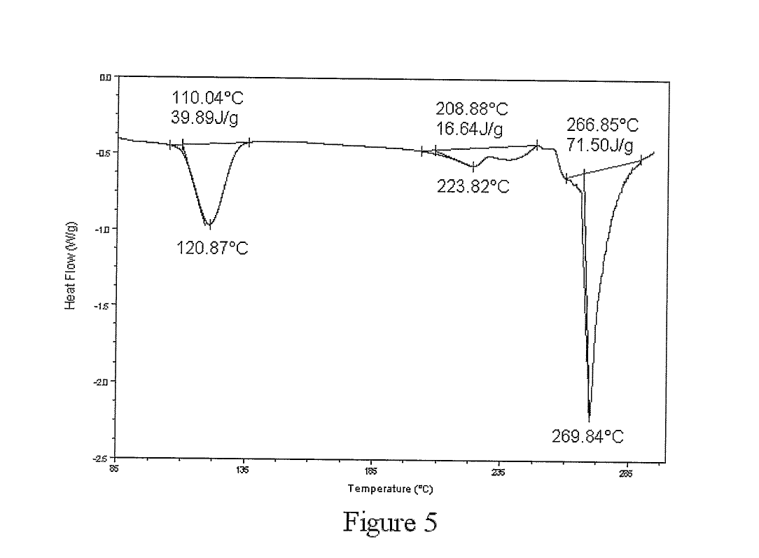

FIG. 5 shows a characteristic DSC curve for the crystalline dodecylsulfate salt anhydrate of Compound I.

A Perkin Elmer model TGA 7 or equivalent instrument was used to obtain the TGA curves. Experiments were performed under a flow of nitrogen and using a heating rate of 10° C./min to a maximum temperature of approximately 250° C. After automatically taring the balance, 5 to 20 mg of sample was added to the platinum pan, the furnace was raised, and the heating program started. Weight/temperature data were collected automatically by the instrument. Analysis of the results was carried out by selecting the Delta Y function within the instrument software and choosing the temperatures between which the weight loss was to be calculated. Weight losses are reported up to the onset of decomposition/evaporation.

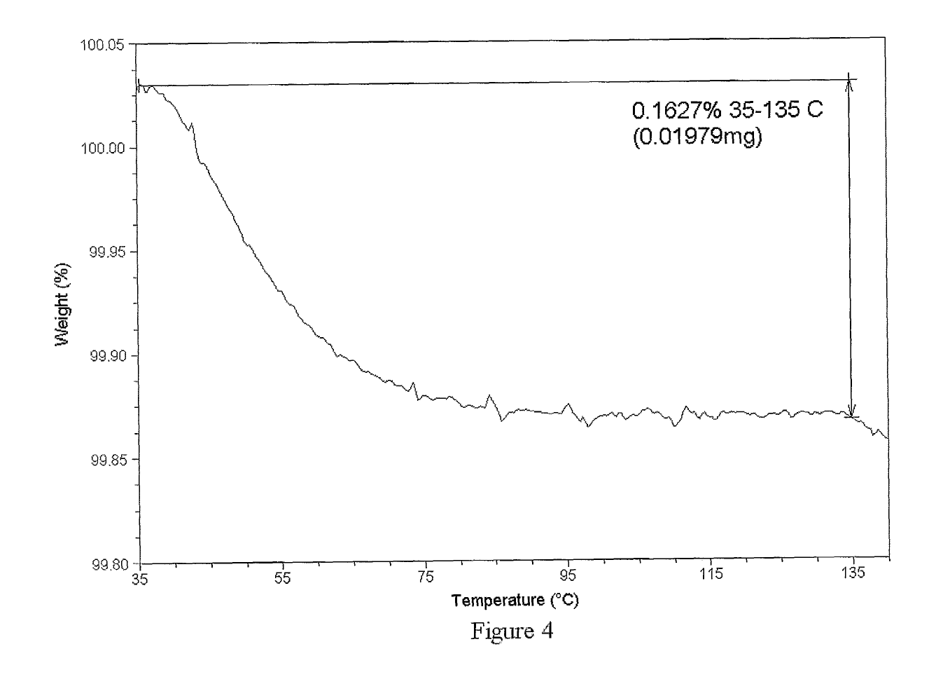

FIG. 4 shows a characteristic thermogravimetric analysis (TGA) curve for the crystalline dodecylsulfate salt anhydrate of Compound I. TGA indicated a weight loss of about 0.2% from ambient temperature to about 135° C.

The crystalline dodecylsulfate salt of the present invention has a phase purity of at least about 5% of the form with the above X-ray powder diffraction and DSC physical characteristics. In one embodiment the phase purity is at least about 10% of the form with the above solid-state physical characteristics. In a second embodiment the phase purity is at least about 25% of the form with the above solid-state physical characteristics. In a third embodiment the phase purity is at least about 50% of the form with the above solid-state physical characteristics. In a fourth embodiment the phase purity is at least about 75% of the form with the above solid-state physical characteristics. In a fifth embodiment the phase purity is at least about 90% of the form with the above solid-state physical characteristics. In a sixth embodiment the crystalline salts of the present invention are the substantially phase pure forms with the above solid-state physical characteristics. By the term “phase purity” is meant the solid state purity of the particular salt with regard to a particular crystalline form of the salt as determined by the solid-state physical methods described in the present application.

### Example of a Pharmaceutical Composition:

The crystalline dodecylsulfate salt of the present invention can be formulated into a tablet by a direct compression process. A 100 mg potency tablet is composed of 100 mg of the active ingredient, 276 mg mannitol, 20 mg of croscarmellose sodium, and 4 mg of magnesium stearate. The active ingredient, microcrystalline cellulose, and croscarmellose are first blended, and the mixture is then lubricated with magnesium stearate and pressed into tablets.

1. A dodecylsulfate salt of (2R)-4-oxo-4-[3-(trifluoromethyl)-5,6-dihydro[1,2,4]triazolo[4,3-a]pyrazin-7(8H)-yl]-1-(2,4,5-trifluorophenyl)butan-2-amine of structural formula I:

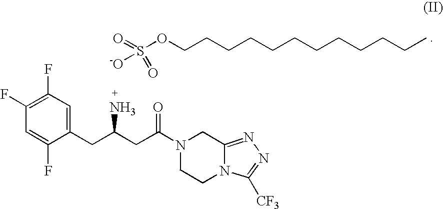

2. The salt of claim 2 characterized in being a crystalline anhydrate.

3. The anhydrate of claim 2 characterized by characteristic absorption bands obtained from the X-ray powder diffraction pattern with 2-theta values of 7.09, 9.46, 10.6, 11.90, 14.11, 17.28, 21.17, 22.92, and 23.87 degrees.

4. The anhydrate of claim 3 further characterized by the X-ray powder diffraction pattern of FIG. 1.

5. The anhydrate of claim 2 characterized by a solid-state carbon-13 CPMAS nuclear magnetic resonance spectrum showing signals at 14.0, 31.8, and 69.0 p.p.m.

6. The anhydrate of claim 5 further characterized by a solid-state carbon-13 CPMAS nuclear magnetic resonance spectrum showing signals at 118.3, 150.5, and 170.2 p.p.m.

7. The anhydrate of claim 6 further characterized by the solid-state carbon-13 CPMAS nuclear magnetic resonance spectrum of FIG. 2.

8. The anhydrate of claim 2 characterized by a solid-state fluorine-19 MAS nuclear magnetic resonance spectrum showing signals at −60.1, −118.7, and −141.3 p.p.m.

9. The anhydrate of claim 8 further characterized by a solid-state fluorine-19 MAS nuclear magnetic resonance spectrum showing signals at −132.9, −93.0, and −20.3 p.p.m.

10. The anhydrate of claim 9 further characterized by the solid-state fluorine-19 MAS nuclear magnetic resonance spectrum of FIG. 3.

11. The anhydrate of claim 2 characterized by the thermogravimetric analysis curve of FIG. 4.

12. The anhydrate of claim 2 characterized by the differential scanning calorimetric curve of FIG. 5.

13. A salt comprising the ions of monoprotonated (2R)-4-oxo-4-[3-(trifluoromethyl)-5,6-dihydro[1,2,4]triazolo[4,3-a]pyrazin-7(8H)-yl]-1-(2,4,5-trifluorophenyl)butan-2-amine cation and dodecylsulfate anion.

14. A pharmaceutical composition comprising a therapeutically effective amount of the salt according to claim 12 in association with one or more pharmaceutically acceptable carriers.

15. (canceled)

16. A method of treating Type 2 diabetes comprising administering to a patient in need of such treatment a therapeutically effective amount of the salt according to claim 1.
